(19) United States
(12) Patent Application Publication
(10) Pub. No.: US 2004/0078034 A1
(43) Pub. Date: Apr. 22, 2004

(54) Coagulator and spinal disk surgery

(76) Inventors: David E Acker, Setauket, NY (US); Todd Fjield, Laguna Hills, CA (US); Edward Paul Harhen, Duxbury, MA (US); Bharat B. Pant, Minneapolis, MN (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/333,087

(22) PCT Filed: Jul. 16, 2001
(86) PCT No.: PCT/US01/22264

(51) Int. Cl. A61B 18/04

(52) U.S. Cl. 606/27

(57) ABSTRACT

An ultrasonic emitting apparatus (10) for cauterizing and ablating tissue. Ultrasonic waves are focused at an intended region of operation by an elongated waveguide (15), thus reducing damage to adjacent unintended tissue. The waveguide (15) can be inserted into structures of the body through overlying organs. The device may be used, for example, to ablate protruding portions of spinal disks.

### CROSS-REFERENCE TO RELATED APPLICATIONS

The present application claims benefit of U.S. Provisional Patent Application No. 60/218,941, the disclosure of which is incorporated by reference herein.

### TECHNICAL FIELD OF THE INVENTION

The present invention relates to ultrasonic instruments usable in medical procedures and to treatment of spinal disk disease using such instruments.

### BACKGROUND ART

A typical electrocautery instrument as shown, for example, in U.S. Pat. No. 5,413,575 incorporates an energy applicator which may be in the form of a blade or pointed probe formed from an electrically conductive metal. The applicator is connected to a source of radio frequency (“RF”) electrical energy. RF energy can be applied onto tissues to be cauterized during surgery as, for example, to stop bleeding from blood vessels cut during surgery. Before applying RF energy, the surgeon must clear away blood or other electrically conductive fluids pooled on the tissue to be cauterized, thus further complicating the procedure. Also, application of RF energy can damage tissues other than the tissue which is intentionally cauterized.

### DISCLOSURE OF THE INVENTION

One aspect of the present invention provides an ultrasonic instrument An instrument according to this aspect of the invention desirably includes a single-use, disposable unit. The disposable unit preferably includes an ultrasonic emitter having a concave emitting surface. The unit also desirably includes a waveguide having a outwardly flaring bell at a rearward end and an elongated tube communicating with the bell and projecting in a forward direction, the bell having an opening at its rearward end. The emitting surface of the emitting element faces into the open end of the waveguide. A substantially gas-free ultrasonic transmission medium most preferably is disposed inside the waveguide and extends from the emitting surface of the ultrasonic emitting element to the forward end of the waveguide.

A unit according to the foregoing aspect of the invention can be connected to a reusable unit which includes a handle. The disposable unit can be connected to the reusable unit to provide a hand-held ultrasonic instrument comprising a handle, an ultrasonic emitting element and a waveguide having an open rearward end and a window at its forward end, the emitting element and the waveguide being mounted to the handle. The reusable unit may include other elements cooperating with the disposable unit, and particularly with the ultrasonic emitter as, for example, a switch for controlling the emitter. Yet another aspect of the invention includes the assembled instrument.

The preferred instruments according to the foregoing aspects of the invention can operate where blood or other fluids are present at a site to be ablated or coagulated. Such instruments can be manually directed at the tissue to be ablated or coagulated in much the same way as a conventional RF electrocautery instrument. The elongated waveguide can be inserted into body structures so that the tip of the waveguide is disposed at a site to be treated. This allows treatment within the body as, for example, ablation of structures covered by other organs. Because the ultrasonic energy is conducted within the waveguide, it need not pass through the organs covering the site to be treated.

Disposable units in accordance with the foregoing aspects of the invention desirably are provided with a gas impermeable package enclosing the emitting element and waveguide, so that the fluid in the waveguide is preserved in a substantially gas-free condition prior to use.

Instruments according to the foregoing aspects of the invention can be used for cauterization and related treatment methods where it is desirable to apply energy to tissues within the body of a subject.

A further aspect of the invention provides methods of alleviating spinal disk disease in a mammalian subject. A method according this aspect of the invention desirably includes the step of inserting an elongated waveguide into a disk to be treated and advancing a tip of the waveguide through the disk until the tip of the waveguide is disposed within the disk, in or adjacent to a portion of the disk protruding into a nerve exit channel of the spine; and delivering ultrasonic energy through the waveguide to the tip so as to ablate tissue within the protruding portion of the disk.

### BRIEF DESCRIPTION OF THE DRAWINGS

FIG. 1 is a diagrammatic exploded view of an instrument according to one embodiment of the invention.

FIG. 2 is a diagrammatic sectional view of a unit used in the instrument of FIG. 1.

### MODES FOR CARRYING OUT THE INVENTION

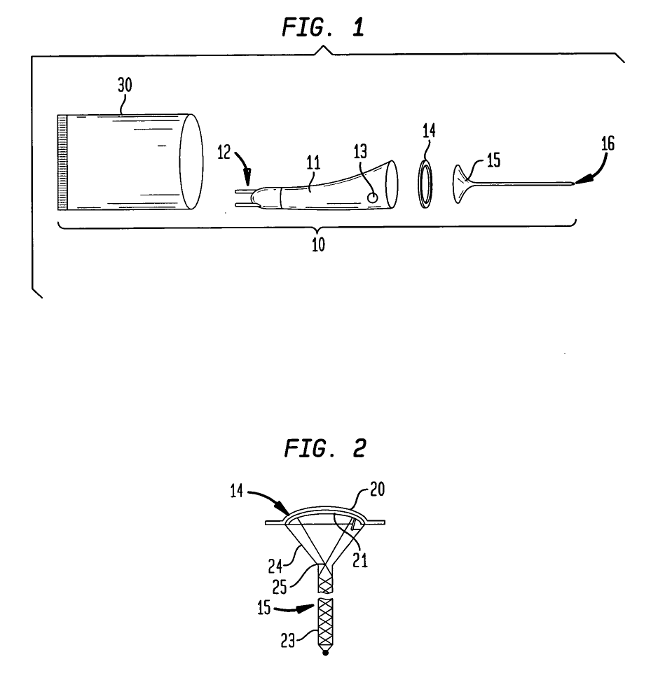

A device according to one aspect of the present invention provides a small, hand-held instrument which can be employed to heat and ablate tissue in a localized region to cauterize, coagulate or ablate this tissue. As seen in exploded view in FIG. 1, the handheld instrument 10 includes a handle 11 adapted to be held by the user. A connector 12 is mounted at the rear end of the handle 11. An actuator button 13 on the exterior of the housing is linked to a switch (not shown) within the interior of the housing. An ultrasonic emitting element 14 is provided at the forward end of the housing (to the right in FIG. 1). As best seen in FIG. 2, the ultrasonic emitting element 14 has an ultrasonic transducer with an active or emitting surface forming the inside of the dome. The ultrasonic emitting element 14 may be a ceramic piezoelectric element having a concave inner surface in the form of a portion of sphere. Such a ceramic piezoelectric element has electrodes on its inner surface and on its outer surface. Alternatively, the ultrasonic emitting element 14 may include a rigid backing such as a metallic backing 20 (FIG. 2) having a concave inner surface and a piezoelectric film element 21 on such inner surface. The piezoelectric film element 21 may include one layer or a plurality of layers of a polymeric piezoelectric material such as PVDF-TrF (polyvinylidene fluoride-trifluoroethylene) copolymer. Where plural layers are provided, the film element desirably has thin metallic electrodes disposed between adjacent layers, and has further electrodes on its emitting surface (facing towards the interior of the dome) and at the backing element 20. The backing element 20 itself may serve as the last-mentioned electrode. Such a polymeric piezoelectric device can be fabricated using the procedures disclosed in copending, commonly assigned U.S. patent application Ser. No. 09/532,614, the disclosure of which is hereby incorporated by reference herein. The electrodes of the piezoelectric element are electrically connected through the switch associated with the actuator button 13 to the connector 12 at the rear of the handle 11. The switch is connected between the connector 12 and the electrodes, or between the connector 12 and the one electrode, so that the electrodes of the ultrasonic emitting element 14 can be connected to or disconnected from the connector 12 by operating the actuator button 13. Typically, the switch is arranged so that the electrodes are normally disconnected from the connector 21, but are connected when the actuator button 13 is depressed by the user.

The apparatus also includes a waveguide 15 incorporating an elongated cylindrical tube 23 and an outwardly flaring element referred to as the “bell” 24. The waveguide 15 is formed from a metal or other rigid material. One preferred set of suitable dimensions for the waveguide 22 consists of a length of 108.75 mm, with the elongated tube 23 of the waveguide 15 being of lenth 90 mm. In conjunction with this length of the waveguide 15 the preferred radius of the bell is 1.01 mm. with a circumference of 20.27 mm. The bell 24 of the waveguide 15 flares outwardly toward the large open end in an exponential fashion That is, the interior surface of the waveguide 15 is in the form of a surface of revolution of an exponential curve around the central axis of the tube 23. As seen schematically in FIG. 2, the open end of the bell 24 faces the emitting surface of the ultrasonic emitting element 14. The dimensions and configuration of the ultrasonic emitting element 14 are selected so that the focus 25 of the ultrasonic emitting element 14 lies just inside the tube 23, at the juncture of the tube 23 and the bell 24. The focus is the point lying at the same distance from all points on the interior or emitting surface of the ultrasonic emitting element 14, i.e., at the center of the spherical emitting surface.

The edge of the bell 24 on the waveguide 15 is sealed to the periphery of the ultrasonic emitting element 14. The interior space within the sealed assembly is filled with a substantially gas-free liquid, gel or other transmission medium having acoustic impedance close to that of water and body tissue. The “HIFU window” 16 or opening at the forward end of the waveguide tube 15 may be covered with a thin membrane (not shown) if necessary to retain the medium within the waveguide. The entire hand-held 10 unit may be provided as disposable, single-use device. The assembly may be packaged within a gas-impermeable envelope or other package 30 to minimize dissolution of air or other atmospheric gases in the medium inside the waveguide 15. Alternatively, the handle 11, actuator button 13, switch and connector 12 may be provided as a reusable unit and the ultrasonic emitting element 14, waveguide 15 and medium may be provided as a single use, disposable item packaged in the same manner. In this case, the disposable unit and the handle 11 are provided with mating fittings adapted to a mechanically engage the ultrasonic emitting element 14 or the waveguide 15 with the handle 11 and to electrically connect the electrodes of the ultrasonic emitting element 14 to the switch and connector 12 when the disposable unit is mated with the handle assembly.

In use, the connector 12 is electrically connected through a flexible cable to a source of electrical excitation signals at an appropriate ultrasonic frequency, as, for example, 1 MHz-5 MHz. The user engages the tip with tissue at a location where cautery, coagulation r ablation is desired as, for example, tissue at the opening of a blood vessel which has been cut during surgery. The user actuates the unit by depressing the actuator button 13 so as to close the switch and direct the excitation signals to the ultrasonic emitting element. Ultrasonic waves generated by the ultrasonic emitting element 14 travel through the medium inside the bell 24 and mutually reinforce one another at the focus 25. The ultrasonic waves propagate down the tube 23 to the HIFU window 16 where they are transmitted into the tissue of the patient. The ultrasonic waves emitted at the window provide concentrated ultrasonic energy which is absorbed by the tissue and rapidly heat the tissue so as to cauterize it. There is no need to remove liquid from the area to be cauterized. Indeed, it is desirable to have at least some liquid surrounding the HIFU window 16 to promote coupling of the ultrasonic energy into the tissue. The concentrated ultrasonic energy is applied only in the immediate vicinity of the HIFU window 16 at a focal point slightly forward of the tip. Surrounding tissues are not heated to any substantial extent and are not damaged by the process. Moreover, there is no electrical current passing through the patients body. Typically, the operator observes the cauterization process and stops the process when cauterization has been achieved.

In further embodiments, the switch inside the handle 11 can be connected to a control connection (not shown) incorporated in the connector 12 so that the switch is electrically connected in a control circuit incorporated in the ultrasonic energy source when the handle 11 is connected to the source. In this arrangement, the terminals of the connector 12 which carry the excitation signal are directly connected to the electrodes of the ultrasonic emitting element 14. The control circuit is arranged to actuate the ultrasonic energy source only when the actuator button 13 is depressed. In a further variant, the control circuit of the ultrasonic energy generator is arranged to deliver a preset dose of ultrasonic energy for each depression of the actuator button 13, for example, by actuating the ultrasonic emitting element 14 for a preselected time with signals of a preselected intensity. The ultrasonic energy source may be equipped with appropriate controls for varying the preselected dose as by varying the preselected time or energy of the signals. In still other embodiments, the actuator button 13 and switch may be omitted and the ultrasonic energy source may be controlled by other control elements as, for example, a foot pedal accessible to the surgeon.

Apparatus of this kind may be utilized for procedures other than cautery as, for example, to ablate undesired tissue which is exposed by surgical procedures.

A further aspect of the invention provides procedures for removing protruding portions of spinal disks. The spinal disks are disposed between the vertebrae of human and other mammalian subjects. The disks can bulge into the relatively narrow nerve exit channels of the vertebrae. In this condition, the disks can bear on the nerves extending from the spinal cord through the nerve exit channels. In a procedure according to this aspect of the invention, the tip of a waveguide 15 is advanced through the disk until the tip of the waveguide 15 is disposed at or adjacent to the portion of the disk protruding into the nerve exit channel. Ultrasonic energy is then supplied through the waveguide 15 to ablate disk tissue within the protruding portion, thereby relieving pressure of the disk on the nerve. Access to the disk may be gained by conventional surgical techniques. For example, in treatment of disk disease of the lumbar region, the waveguide 15 maybe inserted from the posterior side of the spinal column. The waveguide 15 may be inserted into the disks of the cervical spine from the anterior surface, typically through a path obtained by displacing the carotid artery and the esophagus. Ablation of the protruding portion of the disk offers significant advantages over procedures which remove or ablate tissue within the center of the disk. Handheld apparatus incorporating an elongated waveguide 15, such as the apparatus discussed above with reference to FIGS. 1-2 can be used in such a procedure. The elongated waveguide 15 can be inserted readily into the disk and advanced inside the disk into the protruding portion of the disk. This approach minimizes the possibility of inadvertently ablating the nerve or other tissue. The surgeon can determine the proper location of the waveguide tip by feel. However, imaging or other instrumented techniques can be used to aid in this determination. In a variant of this procedure, an instrument other than a waveguide 15 can be positioned as discussed above and actuated to ablate the protruding portion of the disk.

1. A single-use, disposable unit comprising:
(a) an ultrasonic emitter having a concave emitting surface;
(b) a waveguide having an outwardly flaring bell at a rearward end and an elongated tube communicating with the bell and projecting in a forward direction, the bell having an opening at its rearward end, the emitting surface of the emitting element facing into the open end of the waveguide; and
(c) a substantially gas-free ultrasonic transmission medium disposed inside the waveguide and extending from the emitting surface of the ultrasonic emitting element to the forward end of the waveguide.

2. A unit as claimed in claim 1 further comprising a gas impermeable package enclosing the emitting element and waveguide.

3. A hand-held ultrasonic instrument comprising:
(a) a handle;
(b) an ultrasonic emitting element and;
(c) a waveguide having an open rearward end and a window at its forward end, the emitting element and the waveguide being mounted to the handle.

4. A method of alleviating disk disease comprising:
(a) inserting an elongated waveguide into a disk to be treated and advancing a tip of the waveguide through the disk until the tip of the waveguide is disposed within the disk, in or adjacent a portion of the disk protruding into a nerve exit channel of the spine; and
(b) delivering ultrasonic energy through the waveguide to the tip so as to ablate tissue within the protruding portion of the disk.
